(19) United States
(12) Patent Application Publication
(10) Pub. No.: US 2008/0276938 A1
(43) Pub. Date: Nov. 13, 2008

(54) Method and Device For the Treating Sleep Apnea

(75) Inventors: John C. Jeppesen, Ventura, CA (US); Virgil Venditto, Lake Havasu City, AZ (US)

Correspondence Address: RALPH D CHABOT, 2310 E PONDEROSA DR, SUITE 4, CAMARILLO, CA, 93010-4747, US

(73) Assignee: JOHN C. JEPPESEN DMD, INC., Ventura, CA (US)

(21) Appl. No.: 12/161,029

(22) PCT Filed: Jan. 18, 2007
(86) PCT No.: PCT/US2007/060675

§ 371 Date: Jul. 16, 2008

Related U.S. Application Data
(60) Provisional application No. 60/766,433, filed on Jan. 19, 2006.

(51) Int. Cl. A61M 16/00

(52) U.S. Cl. 128/204.18

(57) ABSTRACT

An improved treatment of obstructive sleep apnea is disclosed. A method and various device combinations are presented. The device combinations describe optimal adjustments to properly position a nasal pillow in each of a patient's nares. A novel swivel joint is described to which a nasal pillow is slip-fit over. The swivel joint prevents loss of a proper seal between the nasal pillow and nare which may otherwise occur as a result of head movement. Also presented is an improved obturator mouthpiece design which substantially eliminates air flow through the oral cavity resulting from leakage in the oropharynx.

### CROSS REFERENCE TO RELATED APPLICATIONS

This application claims the benefit of U.S. provisional patent application bearing Ser. No. 60/766,433 filed Jan. 19, 2006, the content of which is hereby incorporated by reference herein in its entirety for all purposes.

### FIELD OF THE INVENTION

The present invention relates to a device and method for treating Obstructive Sleep Apnea Syndrome (OSAS). More particularly, the present invention relates ways to more efficiently maintain airway seals in the nares and oral cavity.

### BACKGROUND OF THE INVENTION

The prior art has discussed various ways for delivery of positive airway pressure (PAP) to a patient while sleeping to prevent OSAS. While prior art utilizing masks covering both the oral and nasal cavities have been developed, these designs are bulky, tend to leak easily and inhibit a wearer's ability to sleep.

More recent developments in the prior art have focused on a combination of obturating the oral cavity and delivering gas such as air via PAP through airway tubes to the nasal cavity through the patient's nares. A platform for supporting both airway tubes is provided and this platform is supported, at least in part, by being connected to a mouthpiece positioned within the patient's oral cavity. A nasal pillow is positioned around each airway tube within a nare to provide a seal between the airway tube and nare thus preventing leakage of the airflow out of the nostrils.

Representative of the prior art for treating OSAS are U.S. Pat. No. 6,012,455 issued to Goldstein and U.S. Pat. No. 6,571,798 issued to Thornton. Both Goldstein and Thornton use a mouthpiece/platform combination to support airway tubes supplying positive airway pressure (PAP) to a patient's nares and both use nasal pillows to obtain a PAP seal within the nares.

It is important to maintain a PAP seal in each nostril in order to treat OSAS. One problem not solved by the prior art is that the PAP seal made by the nasal pillows is often times broken because one or both of the nasal pillows lose complete seal contact with the nasal wall as a patient wearing the equipment changes position while sleeping.

Another issue in treating OSAS is to control the airflow in and out of a patient. Although PAP through the nares is important, air leakage out of the oral cavity can also affect the efficiency of the OSAS treatment.

### BRIEF SUMMARY OF THE INVENTION

The method and devices developed for treatment of OSAS involve a more efficient way to: a) maintain the seal between each nasal pillow and its respective nare; and, b) obturate the oral cavity. While it is preferable to utilize both of my improvements to treat OSAS, either used in combination with other prior art technologies would improve the treatment capability of OSAS, while not being as efficient a treatment combination.

My invention utilizes an oral/nasal platform with a means to support and maintain the platform in position. This means can be: a) a cylindrical support rod which is connected to a mouthpiece worn by a patient; or, b) a strapping mechanism well known in the art that utilizes straps positioned about a patient's head; or, c) any other means to support the platform in proper position. The platform supports a pair of airway delivery tubes having a nasal pillow swivelly connected to an end of a respective airway delivery tube proximal to the nare.

In a preferred embodiment of the supporting means, the support rod is connected to an oral obturating mouthpiece that incorporates a unique design to enhance obturation of the oral cavity as will be discussed later.

The oral platform is comprised of an angled plate which is connected to a base. The platform can be made of separates pieces and then connected to one another or can be made as an integrated piece by a method such as injection molding.

For embodiments utilizing the cylindrical support rod connected to and extending outward from a mouthpiece, the base of the oral platform has a forward face and a rear face having an aperture connecting both faces. The aperture is sized to receive the cylindrical rod so the platform can be slid along the rod. A means to secure the base in position along the cylindrical rod is provided and which would preferably take the form of a set screw although other embodiments can be used to perform the same function which are known in the art and are thus contemplated by this invention.

The angled plate portion of the platform comprises a pair of apertures, one on either angled side. Preferably, the apertures are tapped into the platform according to the patient's physical characteristics such as nasal diameter/shape, size, and angulation of the nares. Airway tubes, capable of delivering PAP to the nares, are threadably engaged to the angled plate portion as will be discussed below. The goal of aperture placement along the angled plate portion is to align the apertures with a respective nare and more preferably, have a common axis of symmetry between a respective aperture and the long axis of the nasal passage of a respective patient's nare.

Angulation is an important concept for my invention. The platform is designed so that the angled plate portion is angled away from a horizontal plane and toward a perpendicular alignment with the patient's nares. This is best accomplished where a separate base portion is used where its top surface is angled so that its distal edge is higher than its proximal edge, relative to a patient's face. Preferably, the degree of angle is determined by precise measurements of the patient and the relative position of the platform along the elongated rod portion.

The angled plate portion is preferably made of a thermoplastic material or another material that permits a slight angular deformation. Besides the angulation mentioned above for the base portion, the angled plate portion may have its degree of angle adjusted angled inward or outward to more precisely align each aperture with a respective nare.

An airway tube is provided to be threaded through the respective aperture located on the platform. The purpose of external threadable engagement with the platform is so that the length of the portion of each airway tube located between the platform and nare can be adjusted to obtain a more precise fit of the nasal pillow within the patient's nare.

Each airway tube has a threaded exterior surface and can be flexible or more preferably, of rigid construction. Rigid construction is preferred because thread count can be higher than with flexible tubes, thus permitting more precise incremental changes in displacement/adjustment into each respective nare.

Another important feature of my invention is that the interior wall of each airway tube has a substantially smooth interior wall surface to enhance a laminar flow capability. The threaded stem portion of each airway tube is threadably engaged to the platform through a respective aperture.

Each airway tube has its end which is proximate to a nare having a swivel joint either integral or operably connected thereto. Preferably, the swivel joint comprises an upper and a lower swivel component having a hollow interior which is substantially of the same diameter as the inner diameter of the airway tube. The swivel joint further comprises apertures that will permit air to flow into and out.

A common nasal pillow design available for commercial purchase has a three rung configuration. My swivel joint design permits a nasal pillow having three rungs to have the bottom two rungs to be slip-fit over and frictionally attach to at least one external circumferential convexity present on my swivel joint.

Once in position on the swivel joint, the orientation of the nasal pillow relative to the airway tube can be altered. This ability to alter orientation permits a wearer to shift positions while sleeping and maintain an air seal between nasal pillow and nare while PAP is delivered.

With the parts of my invention generally described, features of my invention will now be discussed.

One way to improve nasal seal integrity is the ability to adjust the airway tube height from the platform to the nasal pillow. As described earlier, the platform includes a pair of holes that are positioned for alignment with a particular patient's respective nare. These holes, after proper measurement, are drilled, tapped and then threaded. An airway tube for delivery of air to a respective nare is provided for each respective threaded hole. The exterior surface of each airway tube has a threaded male configuration and sized to engage a threaded hole. With such a design, each airway tube can be incrementally extended or retracted relative to the respective nare thus providing an optimal height for seal efficiency. The airway tubes can be constructed either as flexible or the more preferred rigid type which will permit more precise adjustment.

Another feature of my invention is a new configuration which allows the nasal pillow to be slip-fit over a swivel joint rather than slip-fit over the distal end of an air delivery tube or plenum chamber common in the prior art. Attachment to the swivel joint provides greater stability not only for frictional engagement of the pillow to the air delivery system, but also a seal about the interior wall of the nare that is less likely to be broken by patient movement.

For embodiments utilizing a rod/mouthpiece as a supporting means for the platform, another feature of my invention is an improved obturator design. The objective with the obturator design described in this application is to create a pneumatically-intimate oropharyngeal seal. My obturator design is more efficient because it utilizes and optimizes the oropharyngeal seal which has never been addressed in the prior art. The obturator design extends peripheral acrylic borders as necessary, to seal off oral cavity leakage of PAP during either nasal or oropharyngeal pressurization. Through extension of the peripheral borders into key anatomic areas associated with escape of positive airway pressure, an efficient oropharyngeal seal or obturation is created.

Each nasal pillow fits over and on top of the swivel joint. The swivel joint has an upper swivel component and a lower swivel component which is connected to the distal end of the airway tube. The upper swivel component can be designed with either of two different tolerances: loose/free floating or tight/non-floating that will be described in greater detail below.

As can be noted from the above, various combinations can be created. However, at the time of this writing the preferred embodiment would be the improved obturator design used in conjunction with rigid airway tubes using nasal pillows that are snap fit over two external circumferential convexities designed on a swivel joint connected to the distal end of a respective airway tube.

While certain features of my invention can be used separate from one another and still improve the treatment of sleep apnea, the embodiment which would treat the most difficult patient for sleep apnea would encompass all of my features.

### BRIEF DESCRIPTION OF DRAWINGS

FIG. 1 is a perspective view of a preferred embodiment.

FIG. 2 is a front view of the preferred embodiment illustrated in FIG. 1.

FIG. 3 a side view of the preferred embodiment illustrated in FIG. 1.

FIG. 4 is a view taken along line 4-4 of FIG. 3.

FIG. 5 illustrates movement of a nasal pillow upon a swivel joint.

FIG. 6 is a view taken along line 6-6 of FIG. 3.

FIG. 7 is a front view of the base.

FIG. 8 is a right side view of the base.

FIG. 9 is a rear view of the base.

FIG. 10 is a left side view of the base.

FIG. 11 is a top view of the base.

FIG. 12 is a bottom view of the base.

### BEST MODE FOR CARRYING OUT THE INVENTION

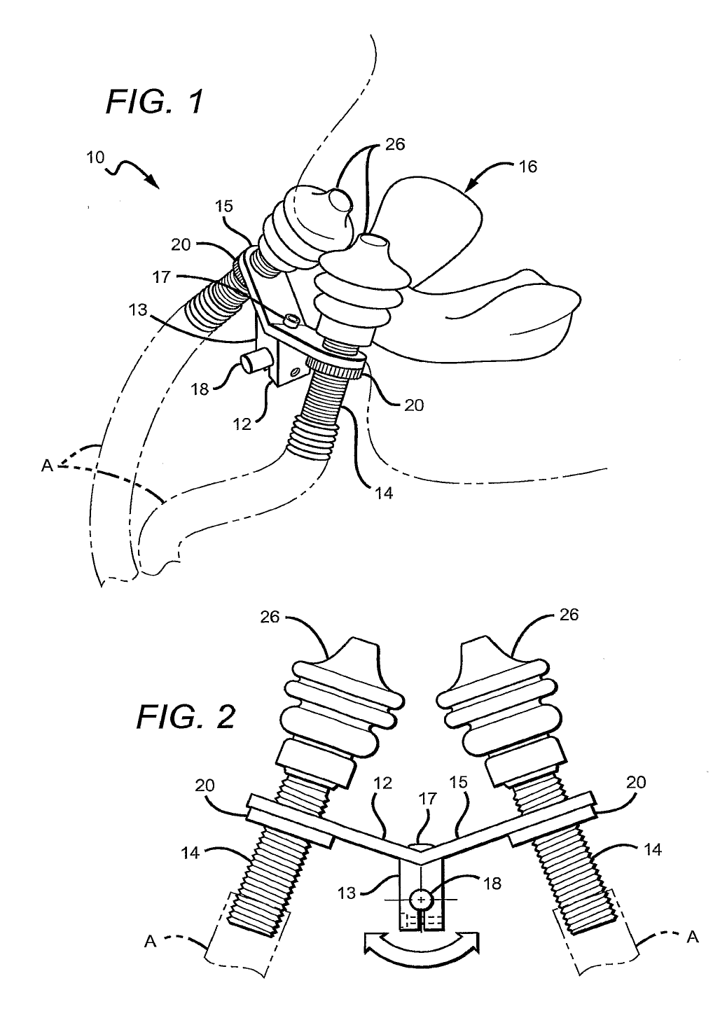

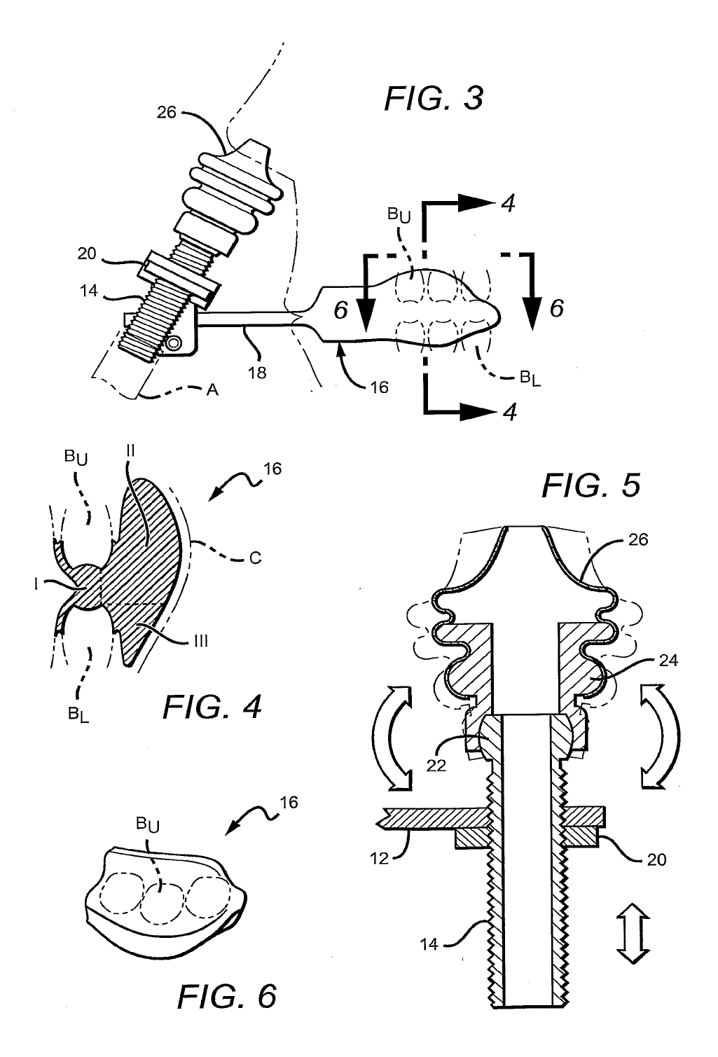

FIG. 1, FIG. 2 and FIG. 3 provide various views of a preferred embodiment 10 for treating OSAS is illustrated. For clarity of illustration, the mouthpiece 16 is not shown in FIG. 2.

Embodiment 10 comprises a tubing retention platform 12 having a base 13 and an angled plate 15, a pair of airway tubes 14 threadably connected to platform 12, and a mouthpiece 16 operably connected to platform 12 for obturating the oral cavity as well as support for platform 12.

Mouthpiece 16 includes an outward extending rod portion 18 upon which platform 12 can be mounted and thereafter incrementally slid to an optimum position from mouthpiece 16 and a patient's nares. Platform 12 can also be pivoted about rod portion 18 as illustrated in FIG. 2.

Rather than having a flat horizontal surface as in the prior art, platform 12 is slightly angled in configuration. This angle improves the alignment of airway tubes 14 to the nares. Proper alignment increases the probability that a PAP seal will not be broken.

There are two types of airway tubing 14 which can be used with my invention.

The first type is a flexible spiral-threaded tube. This spiral-threaded flexible tube may be a plastic material made of polyolefin plastomer. This flexible tube has a male spiral thread on its external surface. This tube would be made via an extrusion process. It has a smooth and flat interior to prevent “rain-out”, and is non-collapsible which prevents crimping. It is important to note that my airway tube design is different than tube designs that were presented in U.S. Pat. No. 6,012,455 and U.S. Patent Publication Number 20050022821 which used “corrugated” plastic tubing. Corrugated tubing has both external and internal corrugations. Besides the chronic tendency to incur detrimental crimping, corrugations were problematic because the internal corrugations have an ability to retain water droplets which are created during the process of air humidification during positive airway pressure (PAP) therapy.

Each flexible spiral airway tube is threaded through a respective aperture created in platform 12. Platform 12 has female threads or countersinks which engage the external male threads on the surface of flexible airway tubing during threading of the two mated parts. Platform 12 is located inferiorly or below the nares of the patient. By turning and/or threading each flexible airway tubing through platform 12, the nares are more precisely approached (superiorly) than was possible with the “corrugated” tubing mechanism of the prior art.

Besides a flexible spiral-threaded tube, the second and most preferred design is a rigid, threaded airway tube. This rigid airway tube is fabricated either by milling stock material or via injection molding. The preferred material to make the rigid, threaded airway tube is sold under the trademark DELRIN®, but may be made of other plasticine materials or some other rigid material like metal. If the part is milled, it would be made by turning on a lathe. Alternatively, DELRIN® or some other plasticine material may be heated, liquefied and subsequently injected into a prefabricated mold.

A rigid threaded airway tube is preferable over flexible threaded tubing for the following two reasons:

a) Increased thread density, i.e., more threads per inch can be created or cut into the exterior surface of a rigid tube than with a flexible spiral tube made via extrusion. Increased thread density allows for more precise adjustment; and,

b) Permits self-seating adjustment of nasal pillow 26 within the nare via movement of the upper swivel component 24. As airway tube 14 is incrementally displaced upward, the swivel joint will swivel into the preferred position by finding the position that minimizes or neutralizes lateral forces within a 360 degree perimeter.

Airway tubes 14 each have a lower end portion and an upper end portion. Each lower end portion frictionally engages tubing “A” and forms a substantially air-tight seal. Tubing “A” supplies PAP from an air supply (not shown).

To engage airway tubes 14 to platform 12, the lower end of airway tube 14 is threaded into a respective hole located on platform 12 until a substantial portion extends below platform 12. Afterwards, lock nut 20 is then threaded from the lower end until it frictionally engages the bottom surface of platform 12. Once frictional engagement of lock nut 20 to platform 12 is made, airway tube 14 can not be displaced unless lock nut 20 is screwed away from engagement with platform 12.

As best illustrated in FIG. 5, the upper end portion of airway tubes 14 comprise a lower swivel component 22 which mates to an upper swivel component 24 so as to form a swivel joint which permits up to a thirty (30) degree compound angle positioning in all directions. It is to be understood that FIG. 5 is displayed for illustration purposes and is not to be interpreted restrictively in that no spacing between components 22 and 24 is present. Components 22 and 24 fit tightly enough to one another that no leakage will occur, preferably to at least 20 cm of water pressure.

The fit between the lower swivel component 22 and upper swivel component 23 can be either: a) loose which would permit the swivel to be “free-floating”; or b) tight, which would require the swivel to be manually adjusted. In other words, by varying the diameter of the lower swivel component 22 convexity, two dynamic tolerances for upper swivel component 24 can be attained. The first tolerance, which preferably is 1000^{th} of an inch smaller than the second tolerance, permits a loose/free floating movement upper swivel component 24. The second tolerance, which is preferably 1000^{th} of an inch larger diameter than the first tolerance, permits a tight/non-floating movement of upper swivel component 24.

Referring to FIG. 5, nasal pillow 26 is positioned about upper swivel component 24 and is frictionally attached to the lower two convexities present on component 24. Further, there are no restrictions present to inhibit airflow from airway tube 14 through lower component 22, upper component 24 and nasal pillow 26.

The nasal pillow/upper swivel component combination is inserted into the patient's nare. If the loose/free floating tolerance is used with the smaller convexity diameter, this unit will move or float with the patient as the patient moves in bed. This movement occurs without manual manipulation of upper swivel component 24. This action helps to maintain a pneumatic seal at the nares during pressurization with patient movement in bed.

When nasal pillows 26 engage the nares, screwing airway tube 14 through platform 12 to displace airway tube 14 toward the nares places an upward force upon upper swivel component 24.

When the free floating or loose design of lower swivel component 22 is used, the upward force discussed above permits upper swivel component 24 to movement in a free-floating manner which in turn induces nasal pillow 26 to determine its optimal position within the nare and become self seating.

Alternatively, when the tolerances between lower swivel component 22 and upper swivel component 24 are minimal so that movement of component 22 relative to component 24 is not free floating, manual adjustment is required to achieve optimal orientation of the nasal pillow within the patient's nare. Once nasal pillow 26 is securely within the nare, manual adjustment of upper swivel component 24 is required to optimize the pneumatic seal. The tight/non-floating embodiment will maintain its position once set manually.

The loose/free floating lower swivel component upper convexity diameter is smaller than the tight/non-floating lower swivel component. The preferred diameter differential between the two described tolerances is 0.001 inches.

Nasal pillow 26 is composed of an elastomeric material well known in the art that will stretch and deform under tension. Upper swivel component 24 holds nasal pillow 26 which is placed about by pulling and stretching nasal pillow 26 into the appropriate position. Upper swivel component 24 has external circumferential convexities that are designed to frictionally engage the interior surface of preferably the lower two rungs of nasal pillow 26. This is unique as nasal pillows are normally retained by grasping the lower constriction from outside the nasal pillow.

FIG. 5 shows an embodiment for nasal pillow 26 having three rungs set in tiered fashion with a nare insert included in the top rung. The largest diameter rung is the middle rung. When compressing the nasal pillow in accordion-like fashion, it is largely the upper rung with the nare insert that is the dynamic component. That is, when nasal pillow 26 is compressed, 95% of the movement occurs in only the top rung. This means that the lower two rungs of the nasal pillow may be used primarily for frictional engagement to upper swivel component 24 avoiding the need to further compress the middle or bottom rung to function properly.

### Optimizing Nare Angulation and Seal During Pressurization

Once each nare is engaged and the swivel angle adjusted, further optimization of the nare seal can be accomplished in three ways:

a) Rotation of platform 12 clockwise or counterclockwise about rod portion 18;

b) Anterior-Posterior (AP) movement of platform 12 along rod portion 18 toward or away from the patient's face; or,

c) deforming platform 12 by heating to bend/deform the right and left wings to accommodate and alter the fixed platform angles.

### Pressurization of the Nares

Once nare angulation and seal appear to be optimized, a pressurization check is performed. Lock nut 20 is threaded on each airway tube 14 as discussed earlier. An air source (not shown) for supplying positive airway pressure in combination with an exhalation port (not shown) is operably connected to the lower portion of each airway tube 14 as is well known in the art. A test is thereafter performed whereby adjustments to the airway tube length between platform 12 and nasal pillow 26 can be made by unscrewing lock nut 20 and adjusting airway tube 14 by either clockwise or counter-clockwise rotation. Once optimum distance (i.e. the distance of airway tube 14 from platform 12 to its respective nare) has been achieved, platform 12 can be further rotated a slight distance about rod portion 18 for a further customization to achieve the most efficient seal of a respective nasal pillow 26 to the nare.

### Design of the Clamp Base

Base 13 is constructed to be integral with angled plate 15 or is made separate and thereafter connected to one another by a hex nut 20 or the like to form platform 12. This base 13 is made of flexible, non-fracturable plastic material. DELRIN® is a preferred material. A substantially round aperture is created anterio-posteriorly through base 13. This aperture allows platform 12 to be slidably mounted upon rod 18. Base 13, and correspondingly platform 12 may be moved toward or away from the patient's face. Below the aperture is a separation space. There is a threaded hole, perpendicular to rod 18, which is provided with a metal screw which is used to secure base 13 to rod 18 as shown in FIG. 1 and FIG. 2.

The optimal lateral width of base 13 (left to right facing the patient) is typically less than 18 mm. This allows the apertures in platform 12 to be closely proximate the midline of the patient's nose, left and right, to accommodate even the narrowest nose. Aperture position allows for the right and left nasal airway tubes 14 (rigid or flexible) to approach a very narrow nose from the correct angle. Having the correct angle is important for achieving a seal at the nares with a nasal pillow.

There are two important angles on the superior surface of base 13. First, there is an anterio-posterior (AP) angle inclination away from the patient's face as measured from a perpendicular from the occlusal plane. Second, there is a lateral, superior wing flare angle, left and right.

The objective is to have the flat surface of the wings of angled plate 15 to be in parallel with angle of the opening of the nares and/or nasal opening inclination. This permits an optimal perpendicular inclination (from this line) of lower swivel component 22. The upper swivel component 24 dynamics are optimized when the ascending direction of the rigid tubes is aligned along the long axis of the nasal passage.

This angle may vary when measured as a relation to the axis of occlusal inclination, the perpendicular approach to the nose will permit the self-seating mechanism if the tolerance of the swivel component is loosely fit to optimize the pneumatic sealing of nasal pillows 26.

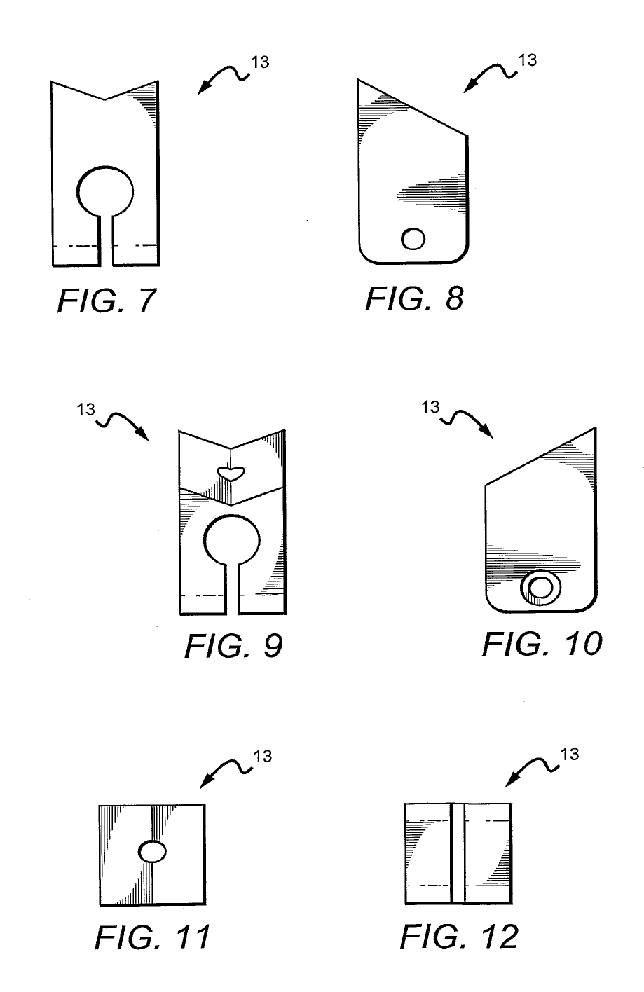

Multiple multi-angled bases 13 can be created to optimize and secure positioning of platform 12. FIG. 7-FIG. 12 illustrate the configuration for base 13. Base 13 designated as 60-20 corresponds to the following:

a) The anterior view shows a twenty (20) degree lateral wing flare angle of platform 12 superiorly (up) right and left.

b) The lateral view shows a 60-degree (AP) inclination away from the perpendicular of the occlusally-parallel rod 18. Again, rod 18 is preferably located parallel to the patient's occlusal plane. Anterior view shows the center of the slide bisecting the midline of the patient's nose.

Another variation of this angulation is in what is termed base 13 designated as 80-5 whereby there is an 80-degree (AP) inclination of base 13 away from the perpendicular of the occlusally-parallel rod 18.

Creating Custom Platform Apertures vs. Standard Widths

Various standard widths for angled plate 15 of platform 12 can be provided. These are predetermined standard widths. For standard widths of angled plate 15, the apertures are pre-drilled before patient examination.

Alternatively, custom nasal width angled plates 15 or platforms 12 can be made for each patient subsequent to examination. Here angled plates 15 having no apertures are initially provided.

Because many noses are not bi-laterally symmetrical, there is need for customization. The long-axis angulation of the left and right nasal passage is determined by the clinician. This angle is then used to select an appropriate PAP Tubing Retention Platform blank angled plate and corresponding base 13. The angled plate is then affixed to base 13 and the center axis hole relating from the nasal angulation determination is located and marked. It is important to document the female-threaded aperture's distance laterally from the center midline of the rod 18. The angle of the approach to the nare is determined by direct line from the respective blank platform wing. The position for the aperture is centered and subsequently drilled and threaded/tapped.

### Centering of Rod 18

It is important to note that there often is a differential between the position of the nasal midline and the maxillary dental midline. Rod 18 needs to be entered under the midline of the nose. By centering rod 18 at the nasal midline, the approach of the left and right tubing toward the nose will be optimized. This measurement corresponds to the naso-maxillary midline. This measurement is useful for properly guiding the mounting of rod 18 onto the anterior surface of the mouthpiece 16.

Rod 18 is milled or injection molded and made of acrylic materials such as clear acrylic or LEXAN®. Rod 18 may be keyed so as to prevent circular rotation of platform 12 via base 13. However, if intentionally left un-keyed, platform 12 can be rotated clockwise or counterclockwise as necessary, to improve nasal angulation and seating of the nasal pillows. This is helpful when the patient's nostrils have less than perfect bilateral symmetry.

### Improvement of the Obturator Design

The original design of the oral cavity obturator described in U.S. Patent Publication number 20050022821 incorporated a clinical philosophy that the tongue will fill intentional voids in the obturator design to complete a pneumatic seal of the oral cavity. In many cases this design will work well for the patient. However, there are some patients who have pneumatically-compromised anatomy. Specifically, there is variability in the relative size of the tongue as compared to other intra-oral structures; or there may be other anatomical anomalies. Where the anatomical variation is such that it contributes to decreased pneumatic seal, an obturator design change is indicated. The objective with the obturator design described in this patent application is to create a pneumatically-intimate oropharyngeal seal.

The obturator has been improved to optimize the oropharyngeal seal. A specific route for venting of PAP has been identified from treating many patients with prior art obturators. Pressurization of the upper airway can occur via the nasal passages or possibly the oropharynx if the nasal passages are restricted. Depending on tongue size, tongue position, and, medial bulging of the adjacent buccal mucosa, due to anatomy and the extent of fat tissue, a certain air pocket-like space will exist or not exist. Some patient's anatomy described above is self-sealing pneumatically. Other patients require pneumatic intervention. Pneumatic intervention is accomplished by redesigning the obturator to obturate more efficiently and/or thoroughly.

As illustrated in FIG. 4, FIG. 5 and FIG. 6, my mouthpiece 16 extends peripheral acrylic borders, as necessary, to seal off oral cavity leakage of PAP between teeth Bu, Bl and cheek “C” during either nasal or oropharyngeal pressurization. Through extension of the peripheral borders into key anatomic areas associated with escape of positive airway pressure, an efficient oropharyngeal seal or obturation is created.

Regarding these certain air pocket-like spaces, these spaces are located within the mouth on the left and right sides of the oral cavity. These spaces also extend superiorly and laterally in both the mandibular and maxillary arches.

In the maxillary arch there is an air pocket-like space which begins at the posterior, inferior base of the zygoma, at the root of the zygoma. This space extends superiorly along the soft mucosal tissue of the vestibule, past the end of the maxillary tuberosity, into the Hamular Notch and out laterally to the buccal mucosa of the cheek. This is the pneumatic seal zone of the maxilla. See FIG. 4.

In the mandibular arch there extends an air pocket-like space inferiorly below the Hamular Notch, down the ramus of the mandible, down to the posterior alveolar ridge and into the posterior buccal mandibular vestibule out laterally as far as the buccal mucosa. FIG. 3 shows a lateral view of this anatomy.

### Method of Impression to Capture Critical Anatomy for Obturator Design

To create the impression involving these air pocket-like spaces, it is preferred to use materials which flow appropriately and correctly in order to accurately capture the anatomy of this vestibule for pneumatic purposes. There are a variety of these materials. A preferred technique is the use of thermo-plastic materials for border-molding such as ADAPTOL® and/or red or green stick dental compound commonly known in the art. Thermo-plastic materials are preferred because they will not overextend the impression capturing this anatomy more accurately.

### Impression of the Maxilla

The material is heated and applied to posterior sections of a custom impression tray. The pliable material is worked up into the vestibule using a finger while the material is warm. The thickness of this border is developed to completely fill this void pneumatically. This technique usually produces thickened, rolled posterior lateral (maxillary) borders on the obturator. Next, while the impression material is still pliable, the mandible is intentionally moved laterally, left and right, to delimit the lateral range of the impression by the patient's upper maxilla and upper mandibular anatomy. Impression of the maxilla may extend as far as the Hamular Notch.

### Impression of the Mandible

The mandibular impression extends into the buccal vestibule posteriorly; additionally contacting the dental-ridge gingiva behind the most posterior mandibular tooth; extending up the ramus of the mandible superiorly and may contact tissue as far up superiorly as the Hamular Notch.

### Figures and Important Pneumatic Zones for Obturator Design

The obturator mouthpiece 16 can be designed to treat patients having: a) a full set of teeth; b) partially-edentulous oral cavities; or, c) completely edentulous oral cavities, using appropriate acrylic extension to fill pneumatic voids.

My improved obturator design focuses on three key anatomic areas which prior art obturator designs have not addressed.

The first area is the soft tissue area directly behind the last posterior teeth left and right, designated as area “T” in FIG. 4. In most cases these would be molar teeth. This is an area where in most patients, the patient's tongue will normally fill this void creating pneumatic integrity. Even behind a third molar is a potential air space void. In the case of a patient with a relatively-small tongue, this void might not have been substantially filled. This type of anatomy will necessitate extensions of the hard acrylic into this pneumatic zone. Specifically, extension of the peripheral posterior borders of the appliance to contact the gingiva of the ascending ramus of the mandible posterior to the last posterior tooth will obturate this first described pneumatic zone.

The second pneumatic anatomic area in discussion is the retro-maxillary vestibule superior to the level of the mandibular posterior teeth, designated as area “II” in FIG. 4. One method is to extend hard acrylic superiorly up to the level of the Hamular Notch and/or laterally and/or further superiorly to the height of the retro-maxillary vestibule fold engaging the buccal mucosa so as to make a slight stretching of this soft tissue fold. The acrylic may be further extended whereby the peripheral border contacts the soft tissue of the ascending ramus of the mandible at or above the level of the mandibular teeth, and further extended whereby the Hamular Notch is contacted.

The portions of mouthpiece 16 related to the retro-maxillary vestibule designated as area “II” is made considering several anatomical factors. There is extension superiorly into the full development of this vestibule, laterally engaging the buccal mucosa, and forward up to the posterior border of the root of zygoma. When engaging the buccal mucosa laterally, the preferred design is to create a buccal convexity, bi-laterally, distal to the first molar teeth. This buccal convexity extension of acrylic will slightly stretch the soft tissue of the cheeks of the patient to improve the dynamics of the oropharyngeal seal.

The third pneumatic anatomic area is designated as area “III” in FIG. 4 and relates to the buccal retro-mandibular vestibule. Here, an extension of acrylic from the mouthpiece extends below the occlusal surface of the mandibular teeth inferiorly and buccally to the depth of the retro-mandibular vestibule. This extension is contiguous laterally to carry the buccal convexity established above in area II such that the lower buccal mucosa is also slightly stretched in a smooth line with the convexity in area II. This area III acrylic extension extends posteriorly from the area of the lower second molar to the ascending ramus of the mandible.

FIG. 3 shows a lateral view of the key anatomical extensions for peripheral acrylic border extension.

In the preferred method for making obturating mouthpiece 16, the dental impression is poured into a model where special recognition is given during pouring of the model to reflect and capture of pneumatic seal areas/zones “I”, “II”, and “III”. The superior, lateral borders of mouthpiece 16 are created by processing of the custom shape on a master model created by using this specific impression technique.

Accordingly, my preferred method for treating sleep apnea would be as follows below.

Patients sometimes require a customized mouthpiece 16 to completely obturate the oral cavity, a necessary requirement. Accordingly, dental impressions of a patient's mouth are preferably taken to identify: i) the space present between the soft tissue area directly behind the last posterior teeth; ii) the retro-maxillary vestibule superior to the level of the occlusal surface of the mandibular posterior teeth; and, iii) the retro-mandibular vestibule inferior to the occlusal surface of the molar teeth and further, posterior to the ascending ramus of the mandible. Obturating mouthpiece 16 is thereafter made from the dental impressions.

Obturating mouthpiece 16 is then connected to the cylindrical elongated rod 18 where the rod extends outward and away from mouthpiece 16 as illustrated in FIG. 3.

An air supply unit (not shown) capable of delivering positive airway pressure through tubing to a patient is provided and will be connected to the airway tubes 14 which are threaded to the angled plate portion 15 of platform 12.

Platform 12 is then slidably mounted upon rod 18. A correct position is then located along rod 18 for the platform's position to be fixed. The correct position of platform 12 is a position where the top surface of angled plate 15 is in substantial perpendicular alignment with the patient's nare.

The threading of the apertures on the angled plate 15 for engagement of the airway tubes 14 typically occurs once the correct position is located.

Each airway tube 14 is then threaded to platform 12 so that a portion of each airway tube 14 extends beneath angled plate 15 and the portion of airway tube 14 having lower swivel component 22 extends above the angled plate 15.

Each of airway tubes 14 are operably connected to an air supply unit (not shown) typically by a tubular connection shown as “A” in FIG. 2 or some other suitable connection means.

Upper swivel component 24 is provided for operably connecting a respective lower swivel component 22. Upper swivel component 24 has at least one circumferential convexity appropriately sized to frictionally attach to nasal pillow 26.

Nasal pillow 26 is frictionally attached to a respective upper swivel component 24, typically by slip-fit. Thereafter, nasal pillows 26 are positioned within a respective nare by first inserting mouthpiece 16 into the patient's mouth to obturate the patient's oral cavity and then adjusting the length of each airway tube 14 extending above the angled plate 15. Finally, the air delivery unit is turned on for delivering PAP to the patient.

1. A device for treating sleep apnea that utilizes a source of positive air pressure being delivered to a patient through the patient's nares using airway tubes supported by a tubing retention platform, and where nasal pillows are positioned relative to respective airway tubes proximal to a respective nare, the improvement comprising:
a means to support and maintain the platform in position;
said platform having a pair of threaded apertures; and,
the airway tubes having a male threaded exterior and substantially smooth interior, the length of the portion of each airway tube located between the tubing retention platform and nare can be threadably adjusted.

2. The device of claim 1 further comprising a swivel joint having a hollow interior and a pair of apertures to permit airflow through said joint, said swivel joint operably connected to an end of a respective airway tube proximate to the nare.

3. The device of claim 2 wherein said swivel joint comprises a lower swivel component connected to the proximal end of a respective airway tube and an upper swivel component swivelly connected to said lower component, said upper component further having at least one circumferential convexity.

4. The device of claim 3 where a respective nasal pillow is positioned about said upper swivel component so that the nasal pillow frictionally attaches to said at least one circumferential convexity.

5. The device of claim 1 where said platform comprises a base having a forward face, a rear face, and an aperture connecting both faces, and where said means to maintain and support the platform comprises a mouthpiece and an elongated rod connected to and extending outward from said mouthpiece, and where said platform base is slidably mounted upon said elongated rod, and a means to secure said base in position upon said elongated rod.

6. The device of claim 5 where said mouthpiece is customized for the patient and designed to create a pneumatically-intimate oropharyngeal seal.

7. The device of claim 6 where said pneumatically-intimate oropharyngeal seal is created by the peripheral borders of the mouthpiece extending into the following anatomic areas of a patient's mouth: i) the space between the soft tissue area directly behind the last posterior teeth; ii) the retro-maxillary vestibule superior to the level of the occlusal surface of the mandibular posterior teeth; and, iii) the buccal retro-mandibular vestibule inferior to the occlusal surface of the molar teeth, and posterior to the ascending ramus of the mandible.

8. The device of claim 1 where said platform comprises an angled plate having a top surface, the angled plate includes said threaded apertures and is configured and orientated where said top surface is perpendicular to a respective patient's nare.

9. The device of claim 8 where each of said threaded apertures has a common axis of symmetry with the long axis of a nasal passage of a respective patient's nare.

10. A method for treating obstructive sleep apnea comprising the steps of:
taking a dental impression of a patient's mouth including: i) the space present between the soft tissue area directly behind the last posterior teeth; ii) the retro-maxillary vestibule superior to the level of the occlusal surface of the mandibular posterior teeth; and, iii) the buccal retro-mandibular vestibule inferior to the occlusal surface of the molar teeth and, posterior to the ascending ramus of the mandible;
making a mouthpiece from said dental impression;
connecting a cylindrical elongated rod to said mouthpiece, where said rod extends outward and away from said mouthpiece;
providing an air supply unit capable of delivering positive airway pressure through tubing to a patient;
providing a platform comprising a base and an angled plate portion positioned upon said base, said base further having a hole appropriately sized for receiving said rod;
providing a pair of airway tubes having an external male-threaded surface each airway tube further comprising a lower swivel component located at the end proximal to be positioned within the patient's nare;
slidably mounting said platform upon said rod;
locating a correct position along said rod for said platform to be fixed;
means for fixing the position of said platform upon said rod;
adjusting the angled plate as necessary so that the top surface of said angled plate is in substantial perpendicular alignment with the patient's nare;
creating a pair of threaded apertures, one aperture associated for each nare, through a respective position on the angled plate of said platform, said apertures appropriately sized to threadably receive the external threads of a respective airway tube;
threadably engaging each of said airway tubes to said platform so that a portion of said airway tube extends beneath said angled plate and the portion of said airway tube having the lower swivel component extends above the angled plate;
operably connecting the end of each of said airway tubes extending below said angled plate to said air supply unit;
providing two nasal pillows;
providing an upper swivel component for operably connection to each of said lower swivel components, said upper swivel component haying at least one circumferential convexity appropriately sized to frictionally attach to a nasal pillow;
operably connecting each of said upper swivel components to a respective lower swivel component;
frictionally attaching each of said nasal pillows to a respective upper swivel component;
thereafter, positioning each of said nasal pillows within a respective nare by first inserting said mouthpiece into the patient's mouth to obturate the patient's oral cavity and then adjusting the length of each airway tube extending above said angled plate; and, operating said air delivery unit.

11. A swivel joint for use in treating a patient with sleep apnea comprising:
a hollow lower component and a hollow upper component swivelly connected to said lower component;
an aperture in said lower component and an operatively connected aperture in said upper component; and,
said upper component having at least one circumferential convexity.

12. The swivel joint of claim 11 where said at least one circumferential convexity is appropriately sized to frictionally attach to a nasal pillow which is positioned about said convexity.

13. The swivel joint of claim 11 having two circumferential convexities.

14. The swivel joint of claim 13 where said two circumferential convexities are appropriately sized to frictionally attach to the inside wall of a nasal pillow which is positioned about said convexities.
